(19) United States
(12) Patent Application Publication
(10) Pub. No.: US 2020/0385776 A1
(43) Pub. Date: Dec. 10, 2020

(54) COMPOSITIONS AND METHODS TO DETECT MICROORGNAISMS

(71) Applicant: 3M INNOVATIVE PROPERTIES COMPANY, St. Paul, MN (US)

(72) Inventors: Kelvin J. Witcher, Hudson, WI (US); Francois Ahimou, Woodbury, MN (US); Naiyong Jing, St. Paul, MN (US); Tonya D. Bonilla, Woodbury, MN (US); Joshua D. Erickson, Champlin, MN (US); Hsi-Chou Liu, Woodbury, MN (US)

(21) Appl. No.: 16/772,588

(22) PCT Filed: Dec. 13, 2018
(86) PCT No.: PCT/IB2018/060062

§ 371 Date: Jun. 12, 2020

Related U.S. Application Data
(60) Provisional application No. 62/607,668, filed on Dec. 19, 2017.

(51) Int. Cl. C12Q 1/04

(52) U.S. Cl. CPC C12Q 1/04

(57) ABSTRACT

A composition, including: amine-modified silica nanoparticles, an indicator compound, and a liquid medium comprising water and no greater than 30 wt-% organic solvent, if present, based on the total weight of liquid medium. A method, including: providing the composition of current application; and contacting a plurality of cells or enzymes with the composition.

### BACKGROUND

Biological indicators used to determine the efficacy of sterilization are well known in the art. Rapid readout biological indicators provide a result in less time compared to their conventional counterparts. The rapid readout is often based on the detection of specific enzyme activities from the test microorganism that correlate with the loss or maintenance of spore viability post-sterilization, which can provide results for the customer within minutes to hours instead of days (conventional growth-based detection) The enzyme activities are often detected using a fluorogenic enzyme substrate where the converted substrate produces a fluorescent compound, that when excited, emits light at a specific wavelength.

### SUMMARY

The present disclosure generally relates to compositions and methods for detecting microorganisms.

In one aspect, the present disclosure provides a composition, comprising: amine-modified silica nanoparticles, an indicator compound, and a liquid medium comprising water and no greater than 30 wt.-% organic solvent, if present, based on the total weight of liquid medium.

In another aspect, the present disclosure provides a method, comprising: providing the composition of current application; and contacting a plurality of cells or enzymes with the composition.

In another aspect, the present disclosure provides an article, comprising: a film; and a dried coating adhered to the film, the coating comprising tertiary amine-modified silica nanoparticles; and a plurality of cells or enzymes.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### BRIEF DESCRIPTION OF DRAWINGS

FIG. 1 is a plan view of one embodiment of an article according to the present disclosure.

FIG. 2 is a side view of the article of FIG. 1.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

As used in this Specification, the recitation of numerical ranges by endpoints includes all numbers subsumed within that range (e.g. 1 to 5 includes 1, 1.5, 2, 2.75, 3, 3.8, 4, and 5, and the like).

Unless otherwise indicated, all numbers expressing quantities or ingredients, measurement of properties and so forth used in the Specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

The present disclosure generally relates to compositions and methods that may be used to detect microorganisms such as bacteria and fungi. In particular, the present disclosure relates to a composition comprising amine-modified silica nanoparticles, an indicator compound, and a liquid medium. Advantageously, composition can significantly increase the reaction velocity (speed of the reaction and/or detection) when in combination with pH 6 or pH 7 liquid medium and decrease time to maximum fluorescence. Accordingly, the method can be used to rapidly detect bacteria.

In one aspect, the present disclosure provides a composition. The composition can include amine-modified silica nanoparticles, an indicator compound, and a liquid medium.

The amine-modified silica nanoparticles can be functionalized with amine groups. Preferably, the amine groups are covalently bonded to one portion (i.e., set) of the population of nanoparticles. Herein, “amine” includes primary or secondary, tertiary and quaternary ammonium. They may be aliphatic or aromatic. Preferably, the amine groups are tertiary amines. In some embodiments, the amine-modified silica nanoparticles are tertiary amine-modified silica nanoparticles having tertiary amine groups attached to the surface through covalent bonds.

Silica nanoparticles of the present invention include silica. Nanoparticles can include essentially only silica (although other oxides can be used, such as ZrO₂, colloidal zirconia, Al₂O₃, colloidal alumina, CeO₂, colloidal ceria, SnO₂, colloidal tin (stannic) oxide, TiO₂, colloidal titanium dioxide), or they can be composite nanoparticles such as core-shell nanoparticles. A core-shell nanoparticle can include a core of an oxide (e.g., iron oxide) or metal (e.g., gold or silver) of one type and a shell of silica (or zirconia, alumina, ceria, tin oxide, or titanium dioxide) deposited on the core. Silica is the most preferred nanoparticle, particularly silica nanoparticles derived from a silicate, such as an alkali metal silicate or ammonium silicate. Herein, “silica nanoparticles” refer to nanoparticles that include only silica as well as core-shell nanoparticles with a surface that includes silica.

The unmodified nanoparticles can be provided as a sol rather than as a powder. Preferred sols generally contain from 15 wt-% to 50 wt-% of colloidal particles dispersed in a fluid medium. Representative examples of suitable fluid media for the colloidal particles include water, aqueous alcohol solutions, lower aliphatic alcohols, ethylene glycol, N,N-dimethylacetamide, formamide, or combinations thereof. The preferred fluid medium is aqueous, e.g., water and optionally one or more alcohols. When the colloidal particles are dispersed in an aqueous fluid, the particles can be stabilized due to common electrical charges that develop on the surface of each particle. The common electrical charges tend to promote dispersion rather than agglomeration or aggregation, because the similarly charged particles repel one another.

Inorganic silica sols in aqueous media are well known in the art and available commercially. Silica sols in water or water-alcohol solutions are available commercially under such trade names as LUDOX (manufactured by E.I. duPont de Nemours and Co., Inc., Wilmington, Del.), NYACOL (available from Nyacol Co., Ashland, Mass.) or NALCO (manufactured by Nalco Chemical Co., Oak Brook, Ill.). Some useful silica sols are NALCO 1115, 2326, 1050, 2327, and 2329 available as silica sols with mean particle sizes of 4 nanometers (nm) to 77 nm. Another useful silica sol is NALCO 1034a available as a silica sol with mean particle size of 20 nanometers. Another useful silica sol is NALCO 2326 available as a silica sol with mean particle size of 5 nanometers. Additional examples of suitable colloidal silicas are described in U.S. Pat. No. 5,126,394.

Suitable silica nanoparticles or used in coating compositions of the present invention can be acicular. The term “acicular” refers to the general needle-like, elongated shape of the particles and may include other sting-like, rod-like, chain-like shapes, as well as filamentary shapes. The acicular colloidal silica particles may have a uniform thickness of 5 to 25 nm, a length, D₁, of 40 to 500 nm (as measured by dynamic light-scattering method) and a degree of elongation D₁/D₂ of 5 to 30, wherein D₂ means a diameter in nm calculated by the equation D₂=2720/S and S means specific surface area in m²/g of the particle, as is disclosed in the specification of U.S. Pat. No. 5,221,497.

U.S. Pat. No. 5,221,497 discloses a method for producing acicular silica nanoparticles by adding water-soluble calcium salt, magnesium salt or mixtures thereof to an aqueous colloidal solution of active silicic acid or acidic silica sol having a mean particle diameter of 3 to 30 nm in an amount of 0.15 to 1.00 wt. % based on CaO, MgO or both to silica, then adding an alkali metal hydroxide so that the molar ratio of SiO₂/M₂O (M:alkali metal atom) becomes 20 to 300, and heating the obtained liquid at 60 to 300° C. for 0.5 to 40 hours. The colloidal silica particles obtained by this method are elongate-shaped silica particles that have elongations of a uniform thickness within the range of 5 to 40 nm extending in only one plane. Acicular silica sol may also be prepared as described in U.S. Pat. No. 5,597,512.

Useful acicular silica particles may be obtained as an aqueous suspension under the trade name SNOWTEX-UP by Nissan Chemical Industries (Tokyo, Japan). The mixture consists of 20-21% (w/w) of acicular silica, less than 0.35% (w/w) of Na₂O, and water. The particles are 9 to 15 nanometers in diameter and have lengths of 40 to 300 nanometers. The suspension has a viscosity of less than 100 mPas at 25° C., a pH of 9 to 10.5, and a specific gravity of 1.13 at 20° C.

Other useful acicular silica particles may be obtained as an aqueous suspension under the trade name SNOWTEX-PS-S and SNOWTEX-PS-M by Nissan Chemical Industries, having a morphology of a string of pearls comprised of nanoparticles. The mixture consists of 20-21% (w/w) of silica, less than 0.2% (w/w) of Na₂O, and water. The SNOWTEX-PS-M particles are 18 to 25 nanometers in diameter and have lengths of 80 to 150 nanometers. The particle size is 80 to 150 nanometers by dynamic light scattering methods. The suspension has a viscosity of less than 100 mPas at 25° C., a pH of 9 to 10.5, and a specific gravity of 1.13 at 20° C. The SNOWTEX-PS-S has a particle diameter of 10-15 nm and a length of 80-120 nm.

Silica nanoparticles that are surface modified in accordance with the present invention comprise nanometer-sized particles. The term “nanometer-sized” refers to particles that are characterized by an average particle size (i.e., the average of the largest dimension of the particles, or the average particle diameter for spherical particles) in the nanometer range, often no greater than 100 nanometers (nm), and preferably no greater than 60 nm (prior to surface modification, i.e., functionalization). More preferably, the average particle size is no greater than 45 nm (prior to surface modification), even more preferably no greater than 20 nm (prior to surface modification), even more preferably no greater than 10 nm (prior to surface modification), and even more preferably no greater than 5 nm (prior to surface modification). Preferably, prior to surface modification, the average particle size of the silica nanoparticles is at least 1 nm, more preferably at least 2 nm, even more preferably at least 3 nm, and even more preferably at least 4 nm, and even more preferably at least 5 nm. A particularly preferred particle size is 4 nm to 5 nm.

Average particle size of the nanoparticles can be measured using transmission electron microscopy. In the practice of the present invention, particle size may be determined using any suitable technique. Preferably, particle size refers to the number average particle size and is measured using an instrument that uses transmission electron microscopy or scanning electron microscopy. Another method to measure particle size is dynamic light scattering that measures weight average particle size. One example of such an instrument found to be suitable is the N4 PLUS SUB-MICRON PARTICLE ANALYZER available from Beckman Coulter Inc. of Fullerton, Calif.

It is also preferable that the nanoparticles be relatively uniform in size. Uniformly sized nanoparticles generally provide more reproducible results. Preferably, variability in the size of the nanoparticles is less than 25% of the mean particle size.

Herein, nanoparticles (prior to functionalization) are water-dispersible to reduce, and preferably prevent, excessive agglomeration and precipitation of the particles in an aqueous environment. If necessary, water-dispersibility can be enhanced by functionalizing the nanoparticles with water-dispersible groups. Nanoparticle aggregation can result in undesirable precipitation, gellation, or a dramatic increase in viscosity; however, small amounts of agglomeration can be tolerated when the nanoparticles are in an aqueous environment as long as the average size of the agglomerates (i.e., agglomerated particles) is no greater than 60 nm. Thus, the nanoparticles are preferably referred to herein as colloidal nanoparticles since they can be individual particles or small agglomerates thereof.

The nanoparticles preferably have a surface area of at least 10 m²/gram, more preferably at least 20 m²/gram, and even more preferably at least 25 m²/gram. The nanoparticles preferably have a surface area of greater than 750 m²/gram.

Nanoparticles of the present invention can be porous or nonporous.

In preferred embodiments, a portion of the nanoparticles of the present invention are modified or functionalized with amine groups, which can be formed typically using aminosiloxane chemistry. The amine groups can be protected if desired. Combinations of protected amine groups and unprotected amine groups can be used if desired.

The amine groups are covalently bonded to a preferred silica surface of individual nanoparticles, preferably through Si—O—Si bonds. Other nanoparticles containing zirconia, alumina, ceria, tin oxide, or titanium dioxide, may similarly be attached to aminosiloxanes by the chemical bonds Zr—O—Si, Al—O—Si, Ce—O—Si, Sn—O—Si, and Ti—O—Si, respectively. These chemical bonds may not be as strong as the siloxane bond, Si—O—Si; however, their bond strength can be enough for the present coating applications.

The level of coverage of the amine-functionalized nanoparticles herein is reported in terms of the concentration of amine groups in the coating composition, assuming 100% of the amount of amine groups in the coating composition would be covalently bonded to surfaces of the silica particles. Preferably, the amine groups are present on a particle surface in the coating composition in an amount equivalent to at least 3 mole-% of the total molar functional groups on said surface.

More preferably, the amine groups are present on a particle surface in the coating composition in an amount equivalent to at least 5 mole-%, even more preferably at least 10 mole-%, and even more preferably at least 25 mole-%, of total molar functional groups on said surface. Higher molar equivalents of amine groups can contribute to more bonds between particles, thereby forming a coating with a more dense particle network. In certain situations, an excess of amine groups (i.e., greater than 100%) can be desirable; however, typically the amount of amine groups are present on a particle surface in the coating composition in an amount equivalent to no more than 150 mole-% of the total molar functional groups on said particle surface. Due to the multifunctionality of the alkoxy aminoalkyl-substituted organosilanes (e.g., 3-(2-aminoethyl)aminopropyl trimethoxysilane), when the coating composition includes more than 100 mole-% amine groups, more than a monolayer of the aminosiloxane is created on the particle surface. An excess of hydrolyzed alkoxy aminoalkyl-substituted organosilane, when present, can also function as a primer on the surface of the substrate.

The nanoparticle functionalization with amine groups can be accomplished using conventional techniques. For silica nanoparticles, however, it has been discovered that reacting alkoxy aminoalkyl- substituted organosilanes (e.g., 3-(2-aminoethyl)aminopropyl trimethoxysilane) to create amino functionality on the surface of the silica nanoparticles can be advantageously accomplished (for example, without gelling) using basic conditions. Preferably, this is accomplished at a pH of at least 10.5, even more preferably at a pH of at least 11.0, even more preferably at a pH of at least 11.5, even more preferably at a pH of at least 12.0, and even more preferably at a pH of at least 12.5. A typically upper pH is 14.0. In a typical method, the pH of an aqueous dispersion of silica nanoparticles is initially adjusted to this pH to generate negatively charged silica particles. Then the alkoxy aminoalkyl-substituted organosilane is combined with the negatively charged silica nanoparticles and allowed to react for a time effective for the alkoxysilyl end of the alkoxy aminoalkyl-substituted organosilane to preferentially react with the negatively charged silica surface. Such pH is maintained for a time effective to cause reaction between the alkoxysilyl end of the alkoxy aminoalkyl-substituted organosilane and the silica nanoparticles. Typically, this is at least 2 hours, preferably at least 8 hours, and more preferably at least 12 hours. Temperatures above room temperature (e.g., 60° C.-80° C.) can be used to reduce the reaction time. The desired pH and time of reaction are ones that enhance functionalization and enhance stability of the composition (e.g., reduce precipitation and/or agglomeration of the particles). After the functionalization reaction is carried out to the desired level (preferably, completed), the pH of the coating solution may be brought to a desired pH (e.g., to a range of 5 to 8).

In some embodiments, the composition can further include a plurality of cells or enzymes. Cells can include prokaryotic and eukaryotic cells, for example, bacteria, archaea, fungi, protists and mammalian cells. In some embodiments, the cells can include spore-forming bacteria. The test microorganisms can be any suitable test microorganisms. Suitable test microorganisms include endospore (spore)-forming bacteria (e.g., a species of the genus Geobacillus or Bacillus), bacteria (e.g. a species from the genus Mycobacterium) and fungal spores (e.g. a species of the genus Aspergillus) that are known in the art. In any embodiment wherein the test microorganisms are spores, the spores can comprise spores of a species of spore-forming bacteria. In any embodiment, the spore-forming bacteria can comprise spores of a species of Geobacillus, Bacillus or Clostridium. In any embodiment, the spores of a species of Geobacillus, Bacillus or Clostridium can comprise spores of Geobacillus stearothermophilus, Bacillus atrophaeus, Bacillus subtilis, Clostridium sporogenes, Geobacillus thermoglucosidasius, Geobacillus kaustophilis, for example.

The enzyme can be any suitable enzyme, for example, α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, plasmin, thrombin, trypsin, calpain, α-mannosidase, β-mannosidase, a-L-fucosidase, leucine aminopeptidase, a-L-arabinofuranoside, cysteine aminopeptidase, valine aminopeptidase, β-xylosidase, α-L-iduronidase, glucanase, cellobioside, cellulase, α-arabinosidase, glycanase, sulfatase, butyrate, glycosidase, arabinoside, and a combination of any two or more of the foregoing enzymes.

Indicator compound can facilitates detection of a metabolic activity of the test cells or microorganisms (e.g., spore). In any embodiment, the metabolic activity can be an enzyme activity. Non-limiting examples of indicator compounds include a chromogenic enzyme substrate, a fluorogenic enzyme substrate, a pH indicator, a redox indicator, a chemiluminescent enzyme substrate, a dye, and a combination of any two or more of the foregoing indicator compounds. Suitable indicator compounds can include, for example, derivatives of coumarin including 7-hydroxycoumarin (umbelliferone) derivatives and 4-methylumbelliferone (7-hydroxy-4-methylcoumarin) derivatives including: 4-methylumbelliferyl alpha-D-glucopyranoside, 4-methylumbelliferyl alpha-D-galactopyranoside, 4-methylumbelliferyl heptanoate, 4-methylumbelliferyl palmitate, 4-methylumbelliferyl oleate, 4-methylumbelliferyl acetate, 4-methylumbelliferylnonanoate, 4-methylumbelliferyl caprylate, 4-methylumbelliferyl butyrate, 4-methylumbelliferyl-beta-D-cellobioside, 4-methylumbelliferyl acetate, 4-methylumbelliferyl phosphate, 4-methylumbelliferyl sulfate, 4-methylumbelliferyl-beta-trimethylammonium cinnamate chloride, 4-methylumbelliferyl-beta-D-N,N′,N″-triacetylchitotriose, 4-methylumbelliferyl-beta-D-xyloside, 4-methylumbelliferyl-N-acetyl-beta-D-glucosaminide, 4-methylumbelliferyl-N-acetyl-alpha-D-glucosaminide, 4-methylumbelliferyl propionate, 4-methylumbelliferyl stearate, 4 methylumbelliferyl-alpha-L-arabinofuranoside, 4-methylumbelliferyl alpha-L-arabinoside; methyl umbelliferyl-beta-D-N,N′-diacetyl chitobioside, 4-methylumbelliferyl elaidate, 4-Methylumbelliferyl-alpha-D-mannopyranoside, 4-methylumbelliferyl-beta-D-mannopyranoside, 4-methylumbelliferyl-beta-D-fucoside, 4-methylumbelliferyl-alpha-L-fucoside, 4-methylumbelliferyl-beta-L-fucoside, 4-methylumbelliferyl-alpha-D-galactoside, 4-methylumbelliferyl-beta-D-galactoside, 4-trifluoromethylumbelliferyl beta-D-galactoside, 4-methylumbelliferyl-alpha-D-glucoside, 4-methylumbelliferyl-beta-D-glucoside, 4-methylumbelliferyl-7,6-sulfo-2-acetamido-2-deoxy-beta-D-glucoside, 4-methylumbelliferyl-beta-D-glucuronide, 6,8-difluor-4-methylumbelliferyl-beta-D-glucuronide, 6,8-difluoro-4-methylumbelliferyl-beta-D-galactoside, 6,8-difluoro-4-methylumbelliferyl phosphate, 6,8-difluoro-4-methylumbelliferyl beta-D-xylobioside, for example. The second substrate can also be derivatives of 7-amido-4-methylcoumarin, including: Ala-Ala-Phe-7-amido-4-methylcoumarin, Boc-Gln-Ala-Arg-7-amido-4-methylcoumarin hydrochloride, Boc-Leu-Ser-Thr-Arg-7-amido-4-methylcoumarin, Boc-Val-Pro-Arg-7-amido-4-methylcoumarin hydrochloride, D-Ala-Leu-Lys-7-amido-4-methylcoumarin, L-Alanine 7-amido-4-methylcoumarin trifluoroacetate salt, L-Methionine 7-amido-4-methylcoumarin trifluoroacetate salt, L-Tyrosine 7-amido-4-methylcoumarin, Lys-Ala-7-amido-4-methylcoumarin dihydrochloride, N-p-Tosyl-Gly-Pro-Arg 7-amido-4-methylcoumarin hydrochloride, N-Succinyl-Ala-Ala-Phe-7-amido-4-methylcoumarin, N-Succinyl-Ala-Ala-Pro-Phe-7-amido-4-methylcoumarin, N-Succinyl-Ala-Phe-Lys 7-amido-4-methylcoumarin acetate salt, N-Succinyl-Leu-Leu-Val-Tyr-7-Amido-4-methylcoumarin, D-Val-Leu-Lys 7-amido-4-methylcoumarin, Fmoc-L-glutamic acid 1-(7-amido-4-methylcoumarin), Gly-Pro-7-amido-4-methylcoumarin hydrobromide, L-Leucine-7-amido-4-methylcoumarin hydrochloride, L-Proline-7-amido-4-methylcoumarin hydrobromide; other 7-hydroxycoumarin derivatives including 3-cyano-7-hydroxycoumarin (3-cyanoumbelliferone), and 7-hydroxycoumarin-3-carboxylic acid esters such as ethyl-7-hydroxycoumarin-3-carboxylate, methyl-7-hydroxycoumarin-3-carboxylate, 3-cyano-4-methylumbelliferone, 3-(4-imidazolyl)umbelliferone; derivatives of fluorescein including: 2′,7′-Bis-(2-carboxyethyl)-5-(and-6-)carboxyfluorescein, 2′,7′-bis-(2-carboxypropyl)-5-(and-6-)-carboxyfluorescein, 5- (and 6)-carboxynaphthofluorescein, Anthofluorescein, 2′,7′-Dichlorofluorescein diacetate, 5(6)-Carboxyfluorescein, 5(6)-Carboxyfluorescein diacetate, 5-(Bromomethyl)fluorescein, 5-(Iodoacetamido)fluorescein, 5-([4,6-Dichlorotriazin-2-yl]amino)fluorescein hydrochloride, 6-Carboxyfluorescein, Eosin Y, Fluorescein diacetate 5-maleimide, Fluorescein-O′-acetic acid, O′-(Carboxymethyl)fluoresceinamide, anthofluorescein, rhodols, halogenated fluorescein; derivatives of rhodamine including: Tetramethylrhodamine, Carboxy tetramethyl-rhodamine, Carboxy-X-rhodamine, Sulforhodamine 101 and Rhodamine B; afluorescamine derivatives; derivatives of benzoxanthene dyes inlcuding: seminaphthofluorones, carboxy-seminaphthofluorones seminaphthofluoresceins, seminaphthorhodafluors; derivatives of cyanine including sulfonated pentamethine and septamethine cyanine.

In another aspect, the present disclosure provides a method. The method can be used to detect bacteria. The method can include a step of providing the composition of any embodiment according to the present disclosure and contacting a plurality of cells or enzymes with the composition.

After contacting the article with an effective amount of the liquid medium, the method comprises the step of analyzing the liquid medium to detect cells or enzymes. Detection of cells or enzymes can be performed using any of a variety of detection techniques that are known in the art including, for example, detection of spore germination, detection of microorganism growth, detection of microorganism reproduction, detection of biological activity of cells or enzymes, for example a metabolic activity (e.g., an enzyme activity, fermentation of a nutrient, an oxidation/reduction reaction), and a combination of any two or more of the foregoing detection techniques. In any embodiment, contacting the article with the liquid medium for a period of time can comprise contacting the article with the liquid medium at a predefined temperature that facilitates a metabolic activity of the test cells or enzymes (e.g., the article can be incubated at a temperature suitable for growth and/or enzyme activity of the cells or enzymes).

In any embodiment of any of the methods of the present disclosure wherein the cells or enzymes comprise spores, analyzing the liquid medium to detect a biological activity of the test microorganisms comprises detecting vegetative cells derived from germination and/or outgrowth of the spores.

In any embodiment of any of the methods of the present disclosure wherein the cells comprise spores, analyzing the liquid medium to detect a biological activity of the test microorganisms comprises detecting an enzyme activity of the spores and/or an enzyme activity of vegetative cells derived from germination and/or outgrowth of the spores. In any embodiment, detecting an enzyme activity comprises detecting an enzyme activity selected from the list of beta-D-galactosidase, beta-D-glucosidase, alpha-D-glucosidase, alkaline phosphatase, acid phosphatase, butyrate esterase, caprylate esterase lipase, chloroamphenicol acetytransferase, catechol-2,3-dioxygenase, myristate lipase, leucine am inopeptidase, valine am inopeptidase, chymotrypsin, phosphohydrolase, alpha-D-galactosidase, alpha-L-arabinofuranosidase, N-acetyl-beta-glucosaminidase, beta-D-cellobiosidase, alanine am inopeptidase, proline am inopeptidase, tyrosine aminopeptidase, phenylalanine aminopeptidase, beta-D-glucuronidase, fatty acid esterase, and a combination of any two or more of the foregoing enzyme.

Analyzing the liquid medium can comprise determining whether an indicator compound changed from a first state to a second state. Analyzing the liquid medium can comprise visually observing the liquid medium for a visible change from a first state to a second state. Alternatively or additionally, analyzing the liquid medium can comprise placing the liquid medium into an instrument to analyze the liquid medium for a change from a first state to a second state. In any embodiment, analyzing the liquid medium can comprise comparing the liquid medium and/or comparing the liquid medium to “control”. Analyzing the liquid medium to detect a biological activity of the test microorganisms can comprise analyzing the liquid medium at a pH between 5 and 9, between 6 and 9, between 7 and 9, between 6 and 8, or between 7 and 8. Analyzing the liquid medium at these pH values can avoid adding sodium carbonate at the end of the assay to raise the pH to >10 for liquid, thus decrease the detection time.

In any embodiment of any of the methods of the present disclosure, contacting the article with the liquid medium for a period of time comprises contacting the article with the liquid medium at a predefined temperature. The predefined temperature may vary according to the test microorganism. Suitable predefined temperatures may include temperatures in the range from about 20 degrees C. to about 80 degrees C., for example. In any embodiment of any of the methods of the present disclosure, contacting the article with the liquid medium for a period of time comprises contacting the article with the liquid medium at a pH between 5 and 9.

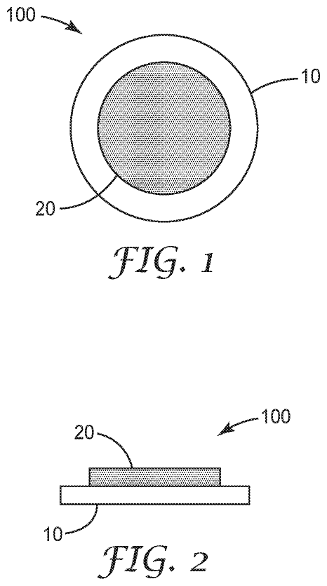

In another aspect, the present disclosure provides an article. FIGS. 1 and 2 show various views of one embodiment of an article 100 according to the present disclosure. The article 100 comprises a film 10 and a dried coating 20 adhered on the substrate. In any embodiment, the film 10 can be a sheet-like material (polypropylene, polyethelene, polyester, polyvinyl chloride films. An example of a suitable sheet-like material for the film 10 is a nonwoven fabric such as, for example, a nonwoven fabric comprising meltblown fibers (e.g., meltblown fibers of a hydrophobic thermoplastic olefin). The coating 20 can include a plurality of cells or enzyme as described above.

The following embodiments are intended to be illustrative of the present disclosure and not limiting.

### EMBODIMENTS

Embodiment 1 is a composition, comprising: amine-modified silica nanoparticles, an indicator compound, and a liquid medium comprising water and no greater than 30 wt-% organic solvent, if present, based on the total weight of liquid medium.

Embodiment 2 is the composition of embodiment 1, further comprising a plurality of cells or enzymes.

Embodiment 3 is the composition of embodiment 2, wherein the cells comprise prokaryotic and eukaryotic cells including bacteria, archaea, fungi, protists and mammalian cells.

Embodiment 4 is the composition of embodiment 2, wherein the cells comprise spore-forming bacteria.

Embodiment 5 is the composition of embodiments 1-4, wherein the indicator compound is selected from the group consisting of a chromogenic enzyme substrate, a fluorogenic enzyme substrate, a pH indicator, a redox indicator, a chemiluminescent enzyme substrate, a dye, and a combination of any two or more of the foregoing indicator compounds.

Embodiment 6 is the composition of embodiments 1-5, wherein the enzyme is selected from the list of consisting of α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, plasmin, thrombin, trypsin, calpain, α-mannosidase, β-mannosidase, α-L-fucosidase, leucine aminopeptidase, a-L-arabinofuranoside, cysteine aminopeptidase, valine aminopeptidase, β-xylosidase, α-L-iduronidase, glucanase, cellobioside, cellulase, α-arabinosidase, glycanase, sulfatase, butyrate, glycosidase, arabinoside, and a combination of any two or more of the foregoing enzymes.

Embodiment 7 is the composition of embodiments 1-6, wherein the amine-modified silica nanoparticles are tertiary amine-modified silica nanoparticles haying tertiary amine groups attached to the surface through covalent bonds.

Embodiment 8 is a method, comprising: providing the composition of embodiments 1-7; and contacting a plurality of cells or enzymes with the composition.

Embodiment 9 is the method of embodiment 8, further comprising analyzing the liquid medium to detect a biological activity of the cells or enzymes.

Embodiment 10 is the method of embodiment 9, wherein analyzing the liquid medium to detect a biological activity of the cells comprises detecting an enzyme activity of bacteria, archaea, fungi, protists or mammalian cells.

Embodiment 11 is the method of embodiment 9, wherein the cells comprise spore-forming bacteria and the enzyme is derived from the spores or from germination and/or outgrowth of a spore.

Embodiment 12 is the method of embodiment 9, wherein detecting an enzyme activity comprises detecting an enzyme activity selected from the list of consisting of α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, plasmin, thrombin, trypsin, calpain, α-mannosidase, β-mannosidase, a-L-fucosidase, leucine aminopeptidase, a-L-arabinofuranoside, cysteine aminopeptidase, valine aminopeptidase, β-xylosidase, α-L-iduronidase, glucanase, cellobioside, cellulase, α-arabinosidase, glycanase, sulfatase, butyrate, glycosidase, arabinoside, and a combination of any two or more of the foregoing enzymes.

Embodiment 13 is the method of embodiments 8-12, wherein analyzing the liquid medium to detect a biological activity of the cells or enzymes comprises analyzing the liquid medium at a pH between 5 and 9.

Embodiment 14 is the method of embodiments 8-13, wherein the composition can comprise a reagent selected from the group consisting of an effective amount of a nutrient that facilitates germination growth or proliferation of the cells, an indicator compound facilitates detection of cell metabolic activity and a combination of any two or more of the foregoing reagents.

Embodiment 15 is an article, comprising: a film; and a dried coating adhered to the film, the coating comprising tertiary amine-modified silica nanoparticles; and a plurality of cells or enzymes.

The following working examples are intended to be illustrative of the present disclosure and not limiting.

### EXAMPLES

Luria-Bertani (LB) broth was prepared by dissolving tryptone (10 g/L), yeast extract (5 g/L), and NaCl (5 g/L) in deionized water. In some examples, 4-methylumbelliferyl-alpha-D-glucopyranoside (0.3 mg/mL) was included in the broth. The pH of the broth was adjusted to either pH 6, pH 7, pH 8, or pH 9 using 0.1 N hydrochloric acid or 1N sodium hydroxide solutions. The broth was passed through a 0.22 micron filter prior to use.

Modified Luria-Bertani (mLB) broth (i.e. without NaCl) was prepared by dissolving tryptone (10 g/L) and yeast extract (5 g/L) in deionized water. The pH of the broth was adjusted to either pH 6, pH 7, pH 8, or pH 9 using 0.1 N hydrochloric acid or 1N sodium hydroxide solutions. The broth was passed through a 0.22 micron filter prior to use.

Tryptone (#BD211705) and yeast extract (#BD212750) were obtained from Becton, Dickinson and Company, Franklin Lakes, N.J.

Deionized water was purified using a MILLI-Q water purification system (EMD Millipore, Burlington, Mass.).

Suspensions of Geobacillus stearothermophilus spores were prepared using molecular biology grade water (#BP2819) obtained from ThermoFischer Scientific, Waltham, Mass.

NALCO 2327 colloidal silica dispersion (20 nm mean particle size, 40 weight percent in water) was obtained from the Nalco Chemical Company, Naperville, Ill.

SILQUEST A-1230 polyethylene oxide trimethoxysilane was obtained from Momentive Performance Materials, Waterford, N.Y.

(N,N-dimethyl-3-aminopropyl)trimethoxysilane was obtained from Gelest Incorporated, Morrisville, Pa.

4-Methylumbelliferone (4-MU) and 4-Methylumbelliferyl-alpha-D-glucopyranoside were obtained from the Sigma-Aldrich Company, St. Louis, Mo. A stock solution of 4-methylumbelliferrone (1 mM) was prepared using molecular using molecular biology grade water (#BP2819) obtained from ThermoFischer Scientific, Waltham, Mass.

alpha-Glucosidase from Geobacillus stearothermophilus was obtained from the Sigma-Aldrich Company, St. Louis, Mo.

### Example 1. Preparation of Amine-Modified Silica Nanoparticle Dispersion

A reaction vessel containing NALCO 2327 dispersion (50 g) was diluted to 20 weight percent with deionized water. SILQUEST A-1230 (1.5 g) was added to the reaction vessel with stirring followed by the addition of (N,N-dimethyl-3-aminopropyl)trimethoxysilane (2.1 g). The reaction vessel was sealed and the reaction mixture was stirred for 16 hours at 80° C. The resulting reaction mixture was sealed in a Spectra/Por 2 dialysis membrane (Spectrum Laboratories, Inc., Rancho Dominguez, Calif.) and then placed in a vessel with continuous flowing water for 16 hours to give the final aqueous dispersion of (N,N-dimethyl-3-aminopropy)trimethoxysilane modified nanosilica particles (mNP).

### Example 2

Detection media was prepared by adding the nanoparticle dispersion of Example 1 (1% volume/volume) to LB broth (pH 6) containing 4-methylumbelliferyl-alpha-D-glucopyranoside (0.3 mg/mL). The detection media (200 microliters per well) was added to two wells of a black 96-well microtiter plate with optically clear, flat bottom wells (NUNC #165301, ThermoFischer Scientific, Waltham, Mass.). A 10 microliter aqueous suspension containing 3×10⁷ Geobacillus stearothermophilus spores (ATCC 7953) was added to each well of the microtiter plate. Next, the contents of each well were mixed using a multiple aspiration/delivery sequence with the pipet. Two control wells were also prepared in which the volume of nanoparticle dispersion (2 microliters) was replaced with an equal volume of molecular biology water (i.e. the control wells did not contain any nanoparticle dispersion). The top of the plate was covered with an optically clear sealing film (product #4311971, Applied Biosystems Corporation, Foster City, Calif.). Fluorescence readings (360 nm excitation/450 nm emission at 50% gain) were taken from the bottom of the plate at 60° C. and without shaking. A reading was taken at an initial time point of 10 seconds and then at +1 minute intervals thereafter. A Synergy Neo2 fluorescence plate reader (BioTek Company, Winooski, Vt.) was used. In Table 1, the mean fluorescence values detected (RLU) at times of 10 seconds, +5 minutes, +10 minutes, +30 minutes, and +60 minutes are reported. (examples 2-4 and the control were all prepared in the same microtiter plate).

### Example 3

The same procedure as described in Example 2 was followed with the exception that the detection media contained a higher concentration of the nanoparticle dispersion of Example 1 (10% volume/volume). In Table 1, the mean fluorescence values detected (RLU) at times of 10 seconds, +5 minutes, +10 minutes, +30 minutes, and +60 minutes are reported. (examples 2-4 and the control were all prepared in the same microtiter plate).

### Example 4

The same procedure as described in Example 2 was followed with the exception that the detection media contained a higher concentration of the nanoparticle dispersion of Example 1 (50% volume/volume). In Table 1, the mean fluorescence values detected (RLU) at times of 10 seconds, +5 minutes, +10 minutes, +30 minutes, and +60 minutes are reported. (examples 2-4 and the control were all prepared in the same microtiter plate).

TABLE 1

| | | Conc. mNP | Measured Fluorescence (RLU) | | | | |
|---|---|---|---|---|---|---|---|
| | pH LB | Dispersion | 10 | +5 | +10 | +30 | +60 |
| | Broth | (% vol/vol) | sec | min | min | min | min |
| Example 2 | 6 | 1 | 9,465 | 14,394 | 15,537 | 16,157 | 16,967 |
| Example 3 | 6 | 10 | 13,782 | 23,050 | 23,826 | 26,414 | 31,161 |
| Example 4 | 6 | 50 | 34,317 | 63,645 | 72,198 | 81,159 | 92,028 |
| Control | 6 | 0 | 7,971 | 13,006 | 14,584 | 14,911 | 15,582 |

### Example 5

Detection media was prepared by adding the nanoparticle dispersion of Example 1 (1% volume/volume) to LB broth (pH 7) containing 4-methylumbelliferyl-alpha-D-glucopyranoside (0.3 mg/mL). The detection media (200 microliters per well) was added to two wells of a black 96-well microtiter plate with optically clear, flat bottom wells (NUNC #165301, ThermoFischer Scientific). A 10 microliter aqueous suspension containing 3×10⁷ Geobacillus stearothermophilus spores (ATCC 7953) was added to each well of the microtiter plate. Next, the contents of each well were mixed using a multiple aspiration/delivery sequence with the pipet. Two control wells were also prepared in which the volume of nanoparticle dispersion (2 microliters) was replaced with an equal volume of molecular biology water (i.e. the control wells did not contain any nanoparticle dispersion). The top of the plate was covered with an optically clear sealing film (product #4311971, Applied Biosystems Corporation). Fluorescence readings (360 nm excitation/450 nm emission at 50% gain) were taken from the bottom of the plate at 60° C. and without shaking. A reading was taken at an initial time point of 10 seconds and then at +1 minute intervals thereafter. A Synergy Neo2 fluorescence plate reader (BioTek Company) was used. In Table 2, the mean fluorescence values detected (RLU) at times of 10 seconds, +5 minutes, +10 minutes, +30 minutes, and +60 minutes are reported. (examples 5-7 and the control were all prepared in the same microtiter plate).

### Example 6

The same procedure as described in Example 5 was followed with the exception that the detection media contained a higher concentration of the nanoparticle dispersion of Example 1 (10% volume/volume). In Table 2, the mean fluorescence values detected (RLU) at times of 10 seconds, +5 minutes, +10 minutes, +30 minutes, and +60 minutes are reported. (examples 5-7 and the control were all prepared in the same microtiter plate).

### Example 7

The same procedure as described in Example 5 was followed with the exception that the detection media contained a higher concentration of the nanoparticle dispersion of Example 1 (50% volume/volume). In Table 2, the mean fluorescence values detected (RLU) at times of 10 seconds, +5 minutes, +10 minutes, +30 minutes, and +60 minutes are reported. (examples 5-7 and the control were all prepared in the same microtiter plate).

TABLE 2

| | | Conc. mNP | | | | | |
|---|---|---|---|---|---|---|---|
| | pH LB | Dispersion | Measured Fluorescence (RLU) | | | | |
| | Broth | (% vol/vol) | 10 sec | +5 min | +10 min | +30 min | +60 min |
| Example 5 | 7 | 1 | 24,284 | 35,771 | 36,656 | 36,647 | 38,884 |
| Example 6 | 7 | 10 | 34,344 | 53,497 | 54,132 | 56,017 | 64,198 |
| Example 7 | 7 | 50 | 39,138 | 78,902 | 96,591 | ≥100,000 | ≥100,000 |
| Control | 7 | 0 | 17,538 | 32,465 | 36,164 | 34,021 | 35,513 |

### Example 8

Detection media was prepared by adding the nanoparticle dispersion of Example 1 (1% volume/volume) to LB broth (pH 8) containing 4-methylumbelliferyl-alpha-D-glucopyranoside (0.3 mg/mL). The detection media (200 microliters per well) was added to two wells of a black 96-well microtiter plate with optically clear, flat bottom wells NUNC #165301, ThermoFischer Scientific). A 10 microliter aqueous suspension containing 3×10⁷ Geobacillus stearothermophilus spores (ATCC 7953) was added to each well of the microtiter plate. Next, the contents of each well were mixed using a multiple aspiration/delivery sequence with the pipet. Two control wells were also prepared in which the volume of nanoparticle dispersion (2 microliters) was replaced with an equal volume of molecular biology water (i.e. the control wells did not contain any nanoparticle dispersion). The top of the plate was covered with an optically clear sealing film (product #4311971, Applied Biosystems Corporation). Fluorescence readings (360 nm excitation/450 nm emission at 50% gain) were taken from the bottom of the plate at 60° C. and without shaking. A reading was taken at an initial time point of 10 seconds and then at +1 minute intervals thereafter. A Synergy Neo2 fluorescence plate reader (BioTek Company) was used. In Table 3, the mean fluorescence values detected (RLU) at times of 10 seconds, +5 minutes, +10 minutes, +30 minutes, and +60 minutes are reported. (examples 8-10 and the control were all prepared in the same microtiter plate).

### Example 9

The same procedure as described in Example 8 was followed with the exception that the detection media contained a higher concentration of the nanoparticle dispersion of Example 1 (10% volume/volume). In Table 3, the mean fluorescence values detected (RLU) at times of 10 seconds, +5 minutes, +10 minutes, +30 minutes, and +60 minutes are reported. (examples 8-10 and the control were all prepared in the same microtiter plate).

### Example 10

The same procedure as described in Example 8 was followed with the exception that the detection media contained a higher concentration of the nanoparticle dispersion of Example 1 (50% volume/volume). In Table 3, the mean fluorescence values detected (RLU) at times of 10 seconds, +5 minutes, +10 minutes, +30 minutes, and +60 minutes are reported. (examples 8-10 were all prepared in the same microtiter plate).

TABLE 3

| | | Conc. mNP | | | | | |
|---|---|---|---|---|---|---|---|
| | pH LB | Dispersion | Measured Fluorescence (RLU) | | | | |
| | Broth | (% vol/vol) | 10 sec | +5 min | +10 min | +30 min | +60 min |
| Example 8 | 8 | 1 | 44,655 | 77,444 | 85,211 | 84,450 | 84,184 |
| Example 9 | 8 | 10 | 41,255 | 80,898 | 95,287 | 98,897 | ≥100,000 |
| Example 10 | 8 | 50 | 46,555 | 90,019 | ≥100,000 | ≥100,000 | ≥100,000 |
| Control | 8 | 0 | 46,225 | 73,327 | 84,338 | 82,840 | 81,399 |

### Example 11

Detection media was prepared by adding the nanoparticle dispersion of Example 1 (1% volume/volume) to LB broth (pH 9) containing 4-methylumbelliferyl-alpha-D-glucopyranoside (0.3 mg/mL). The detection media (200 microliters per well) was added to two wells of a black 96-well microtiter plate with optically clear, flat bottom wells (NUNC #165301, ThermoFischer Scientific). A 10 microliter aqueous suspension containing 3×10⁷ Geobacillus stearothermophilus spores (ATCC 7953) was added to each well of the microtiter plate. Next, the contents of each well were mixed using a multiple aspiration/delivery sequence with the pipet. Two control wells were also prepared in which the volume of nanoparticle dispersion (2 microliters) was replaced with an equal volume of molecular biology water (i.e. the control wells did not contain any nanoparticle dispersion). The top of the plate was covered with an optically clear sealing film film (product #4311971, Applied Biosystems Corporation). Fluorescence readings (360 nm excitation/450 nm emission at 50% gain) were taken from the bottom of the plate at 60° C. and without shaking. A reading was taken at an initial time point of 10 seconds and then at +1 minute intervals thereafter. A Synergy Neo2 fluorescence plate reader (BioTek Company) was used. In Table 4, the mean fluorescence values detected (RLU) at times of 10 seconds, +5 minutes, +10 minutes, +30 minutes, and +60 minutes are reported. (examples 11-13 and the control were all prepared in the same microtiter plate).

### Example 12

The same procedure as described in Example 11 was followed with the exception that the detection media contained a higher concentration of the nanoparticle dispersion of Example 1 (10% volume/volume). In Table 4, the mean fluorescence values detected (RLU) at times of 10 seconds, +5 minutes, +10 minutes, +30 minutes, and +60 minutes are reported. (examples 11-13 and the control were all prepared in the same microtiter plate).

### Example 13

The same procedure as described in Example 11 was followed with the exception that the detection media contained a higher concentration of the nanoparticle dispersion of Example 1 (50% volume/volume). In Table 4, the mean fluorescence values detected (RLU) at times of 10 seconds, +5 minutes, +10 minutes, +30 minutes, and +60 minutes are reported. (examples 11-13 and the control were all prepared in the same microtiter plate).

TABLE 4

| | | Conc. mNP | Measured Fluorescence (RLU) | | | | |
|---|---|---|---|---|---|---|---|
| | pH LB | Dispersion | 10 | +5 | +10 | +30 | +60 |
| | Broth | (% vol/vol) | sec | min | min | min | min |
| Example 11 | 9 | 1 | 30,288 | 63,503 | 85,978 | ≥100,000 | ≥100,000 |
| Example 12 | 9 | 10 | 24,128 | 56,260 | 84,634 | ≥100,000 | ≥100,000 |
| Example 13 | 9 | 50 | 36,746 | 90,197 | ≥100,000 | ≥100,000 | ≥100,000 |
| Control | 9 | 0 | 30,404 | 61,080 | 85,476 | ≥100,000 | ≥100,000 |

### Example 14

A solution (80 microliters) of Luria-Bertani (LB) broth (pH 8) containing 0.3 mg/mL of 4-methylumbelliferyl-alpha-D-glucopyranoside was added to two wells of a black 96-well microtiter plate with optically clear, flat bottom wells (NUNC #165301, ThermoFischer Scientific) followed by the addition of 20 microliters of the nanoparticle dispersion of Example 1 to each well. Five microliters of an alpha-glucosidase from Geobacillus stearothermophilus (125 U/mL) solution in molecular biology grade water was added by pipet to each well and then the contents were mixed using a multiple aspiration/delivery sequence with the pipet. Two control wells were also prepared in which the 20 microliters of nanoparticle dispersion was replaced with 20 microliters of molecular biology water (i.e. the control wells did not contain any nanoparticle dispersion). The top of the plate was covered with an optically clear sealing film (product #4311971, Applied Biosystems Corporation). Fluorescence readings (360 nm excitation/450 nm emission at 50% gain) were taken at 1 minute intervals for 60 minutes from the top of the plate with a Synergy Neo2 fluorescence plate reader (BioTek Company) at 60° C. The plate was shaken for 2 seconds prior to each reading. In Table 5, the mean maximum fluorescence detected (RLU) is reported (examples 14-17 and the individual controls were all prepared in the same microtiter plate).

### Example 15

The same procedure as described in Example 14 was followed with the exception a lower concentration of the alpha-glucosidase from Geobacillus stearothermophilus (62.5 U/mL) solution was used. Two control wells were also prepared in which the 20 microliters of nanoparticle dispersion was replaced with 20 microliters of molecular biology water (i.e. the control wells did not contain any nanoparticle dispersion). In Table 5, the mean maximum fluorescence detected (RLU) is reported (examples 14-17 and the individual controls were all prepared in the same microtiter plate).

### Example 16

The same procedure as described in Example 14 was followed with the exception a lower concentration of the alpha-glucosidase from Geobacillus stearothermophilus (31.25 U/mL) solution was used. Two control wells were also prepared in which the 20 microliters of nanoparticle dispersion was replaced with 20 microliters of molecular biology water (i.e. the control wells did not contain any nanoparticle dispersion). In Table 5, the mean maximum fluorescence detected (RLU) is reported. (examples 14-17 and the individual controls were all prepared in the same microtiter plate).

### Example 17

The same procedure as described in Example 14 was followed with the exception a lower concentration of the alpha-glucosidase from Geobacillus stearothermophilus (15.625 U/mL) solution was used. Two control wells were also prepared in which the 20 microliters of nanoparticle dispersion was replaced with 20 microliters of molecular biology water (i.e. the control wells did not contain any nanoparticle dispersion). In Table 5, the mean maximum fluorescence detected (RLU) is reported. (examples 14-17 and the individual controls were all prepared in the same microtiter plate).

TABLE 5

| | Conc. mNP | | |
|---|---|---|---|
| | Dispersion | alpha- | Maximum |
| | (% vol/vol) | Glucosidase | Fluoresence (RLU) |
| Example 14 | 20 | 0.63 U | 73,747 |
| Control for | 0 | 0.63 U | 58,415 |
| Example 14 | | | |
| Example 15 | 20 | 0.31 U | 69,601 |
| Control for | 0 | 0.31 U | 56,922 |
| Example 15 | | | |
| Example 16 | 20 | 0.16 U | 62,803 |
| Control for | 0 | 0.16 U | 55,794 |
| Example 16 | | | |
| Example 17 | 20 | 0.08 U | 69,323 |
| Control for | 0 | 0.08 U | 54,394 |
| Example 17 | | | |

### Example 18

Modified Luria-Bertani (mLB) broth (pH 6) was added to two wells (100 microliters added per well) of a black 96-well microtiter plate with optically clear, flat bottom wells (Nunc #165301, ThermoFischer Scientific) followed by the addition of 100 microliters of the nanoparticle dispersion of Example 1 to each well. Five microliters of the 4-methylumbelliferone (1 mM) stock solution in molecular biology grade water was added by pipet to each well and then the contents were mixed using a multiple aspiration/delivery sequence with the pipet. Two control wells were also prepared in which the 100 microliters of nanoparticle dispersion was replaced with 100 microliters of molecular biology water (i.e. the control wells did not contain any nanoparticle dispersion). The top of the plate was covered with an optically clear sealing film (product #4311971, Applied Biosystems Corporation). Fluorescence readings (330 nm excitation/450 nm emission at 50% gain) were taken at 1 minute intervals from the bottom of the plate with a Synergy Neo2 fluorescence plate reader (BioTek Company) at 60° C. In Table 6, the mean fluorescence values detected (RLU) at times of 1, 30, and 60 minutes are reported. (examples 18-20 and the individual controls were all prepared in the same microtiter plate).

### Example 19

The same procedure as described in Example 18 was followed with the exception that the mLB broth was adjusted to pH 7 and fluorescence readings were taken with 360 nm excitation/450 nm emission at 50% gain. In Table 6, the mean fluorescence values detected (RLU) after reaction times of 1, 30, and 60 minutes are reported. (examples 18-20 and the individual controls were all prepared in the same microtiter plate).

### Example 20

The same procedure as described in Example 18 was followed with the exception that the mLB broth was adjusted to pH 8 and fluorescence readings were taken with 360 nm excitation/450 nm emission at 50% gain. In Table 6, the mean fluorescence values detected (RLU) after reaction times of 1, 30, and 60 minutes are reported. (examples 18-20 and the individual controls were all prepared in the same microtiter plate).

TABLE 6

| | | Measured | | |
|---|---|---|---|---|
| | pH of | Fluorescence (RLU) | | |
| | mLB Broth | 1 minute | 30 minutes | 60 minutes |
| Example 18 | 6 | 16,341 | 15,635 | 15,707 |
| Control for | 6 | 15,645 | 14,625 | 14,475 |
| Example 18 | | | | |
| Example 19 | 7 | 21,084 | 22,283 | 25,364 |
| Control for | 7 | 9,830 | 7,550 | 7,287 |
| Example 19 | | | | |
| Example 20 | 8 | 32,617 | 33,206 | 37,389 |
| Control for | 8 | 31,905 | 23,931 | 22,733 |
| Example 20 | | | | |

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A composition, comprising:
amine-modified silica nanoparticles,
an indicator compound, and
a liquid medium comprising water and no greater than 30 wt-% organic solvent, if present, based on the total weight of liquid medium.

2. The composition of claim 1, further comprising a plurality of cells or enzymes.

3. The composition of claim 2, wherein the cells comprise prokaryotic and eukaryotic cells including bacteria, archaea, fungi, protists and mammalian cells.

4. The composition of claim 2, wherein the cells comprise spore-forming bacteria.

5. The composition of claim 1, wherein the indicator compound is selected from the group consisting of a chromogenic enzyme substrate, a fluorogenic enzyme substrate, a pH indicator, a redox indicator, a chemiluminescent enzyme substrate, a dye, and a combination of any two or more of the foregoing indicator compounds.

6. The composition of claim 1, wherein the enzyme is selected from the list of consisting of α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, plasmin, thrombin, trypsin, calpain, α-mannosidase, β-mannosidase, a-L-fucosidase, leucine aminopeptidase, a-L-arabinofuranoside, cysteine aminopeptidase, valine aminopeptidase, β-xylosidase, α-L-iduronidase, glucanase, cellobioside, cellulase, α-arabinosidase, glycanase, sulfatase, butyrate, glycosidase, arabinoside, and a combination of any two or more of the foregoing enzymes.

7. The composition of claim 1, wherein the amine-modified silica nanoparticles are tertiary amine-modified silica nanoparticles having tertiary amine groups attached to the surface through covalent bonds.

8. A method, comprising:
providing the composition of claim 1; and
contacting a plurality of cells or enzymes with the composition.

9. The method of claim 8, further comprising analyzing the liquid medium to detect a biological activity of the cells or enzymes.

10. The method of claim 9, wherein analyzing the liquid medium to detect a biological activity of the cells comprises detecting an enzyme activity of bacteria, archaea, fungi, protists or mammalian cells.

11. The method of claim 9, wherein the cells comprise spore-forming bacteria and the enzyme is derived from the spores or from germination and/or outgrowth of a spore.

12. The method of claim 9, wherein detecting an enzyme activity comprises detecting an enzyme activity selected from the list of consisting of α-glucosidase, α-galactosidase, lipase, esterase, acid phosphatase, alkaline phosphatase, proteases, aminopeptidase, chymotrypsin, β-glucosidase, β-galactosidase, α-glucoronidase, β-glucoronidase, phosphohydrolase, plasmin, thrombin, trypsin, calpain, α-mannosidase, β-mannosidase, a-L-fucosidase, leucine aminopeptidase, a-L-arabinofuranoside, cysteine aminopeptidase, valine aminopeptidase, β-xylosidase, α-L-iduronidase, glucanase, cellobioside, cellulase, α-arabinosidase, glycanase, sulfatase, butyrate, glycosidase, arabinoside, and a combination of any two or more of the foregoing enzymes.

13. The method of claim 8, wherein analyzing the liquid medium to detect a biological activity of the cells or enzymes comprises analyzing the liquid medium at a pH between 5 and 9.

14. The method of claim 8, wherein the composition can comprise a reagent selected from the group consisting of an effective amount of a nutrient that facilitates germination growth or proliferation of the cells, an indicator compound facilitates detection of cell metabolic activity and a combination of any two or more of the foregoing reagents.

15. An article, comprising:
a film; and
a dried coating adhered to the film, the coating comprising
tertiary amine-modified silica nanoparticles; and
a plurality of cells or enzymes.
